(19) United States
(12) Patent Application Publication
(10) Pub. No.: US 2018/0274534 A1
(43) Pub. Date: Sep. 27, 2018

(54) PUMPING SYSTEM

(71) Applicants: William Fred Wiedemann, III, Livermore, CA (US); Linda Rayburn, San Clemente, CA (US); Eric Phillip Wiedemann, Livermore, CA (US); William Fred Wiedemann, JR., Carlsbad, CA (US)

(72) Inventors: William Fred Wiedemann, III, Livermore, CA (US); Linda Rayburn, San Clemente, CA (US); Eric Phillip Wiedemann, Livermore, CA (US); William Fred Wiedemann, JR., Carlsbad, CA (US)

(21) Appl. No.: 15/935,710

(22) Filed: Mar. 26, 2018

Related U.S. Application Data
(60) Provisional application No. 62/476,453, filed on Mar. 24, 2017.

(51) Int. Cl. F04B 49/20; F04B 43/12; F04B 49/06; A61B 17/3203

(52) U.S. Cl. CPC F04B 49/20; F04B 43/1253; A61B 2017/00539; A61B 17/3203; F04B 49/065

(57) ABSTRACT

A pumping system assembly is configured to adjust a flow rate of a liquid in a hydro dermabrasion system. The pumping system assembly includes a peristaltic pump. A tube is joined to a first opening on the peristaltic pump with a first line connector and joined to a second opening on the peristaltic pump with the second line connector. A hydro dermabrasion handpiece is configured to administer the liquid and connected to the first line connector with a first line. A liquid reservoir is configured to store the liquid for the hydro dermabrasion system and connected to the second line connector with a second line. Optionally a microcontroller is connected to the peristaltic pump and programmed with instructions to engage the rotor in a clockwise manner drawing liquid from the liquid reservoir and transferring the liquid to the hydro dermabrasion handpiece. Optionally a microcontroller is connected to the peristaltic pump and programmed with instructions to engage the rotor in a counter-clockwise manner drawing liquid from the fluid delivery system and transferring the liquid to the liquid reservoir.

### RELATED APPLICATION

This application claims priority to provisional patent application U.S. Ser. No. 62/476,453 filed on Mar. 24, 2017 the entire contents of which is herein incorporated by reference.

### BACKGROUND

The embodiments herein relate generally to pumping systems.

Prior to embodiments of the disclosed invention, flow rate of a hydro dermabrasion system was difficult to effectively control. The vacuum system was used to draw fluid to the handpiece. Indeterminate solution flow to a water dermabrasion hand piece wasted facial treatment solution. By deterministically controlling the treatment fluid flow rate, solution can be used more effectively creating both more effectivity and less waste. By rapidly filling the fluid delivery lines prior to treatment less time would be needed for the treatment. By returning the unused sterile solution that is in the fluid delivery lines back to the solution reservoir after treatment, less solution would be wasted. Embodiments of the disclosed invention solve these problems.

### SUMMARY

A pumping system assembly is configured to adjust a flow rate of a liquid in a hydro dermabrasion system. The pumping system assembly includes a peristaltic pump. A tube is joined to a first opening on the peristaltic pump with a first line connector and joined to a second opening on the peristaltic pump with the second line connector. A hydro dermabrasion handpiece is configured to administer the liquid and connected to the first line connector with a first line. A liquid reservoir is configured to store the liquid for the hydro dermabrasion system and connected to the second line connector with a second line. Optionally, a microcontroller is connected to the peristaltic pump and programmed with instructions to engage the rotor in a clockwise manner drawing liquid from the liquid reservoir and transferring the liquid to the hydro dermabrasion handpiece. Optionally, a microcontroller is connected to the peristaltic pump and programmed with instructions to engage the rotor in a counter-clockwise manner drawing liquid from the fluid delivery system and transferring the liquid to the liquid reservoir.

### BRIEF DESCRIPTION OF THE FIGURES

The detailed description of some embodiments of the invention is made below with reference to the accompanying figures, wherein like numerals represent corresponding parts of the figures.

FIG. 1 shows a schematic top section view of one embodiment of the present invention; and

FIG. 2 shows a schematic top section view of one embodiment of the present invention.

### DETAILED DESCRIPTION OF CERTAIN EMBODIMENTS

By way of example, one embodiment of the pumping system assembly 10 is configured to adjust a flow rate of a liquid 12 in a hydro dermabrasion system 14. The pumping system assembly 12 comprises a peristaltic pump 20.

The peristaltic pump 20 further comprises a tube pathway 22 set between a first opening 24 and a second opening 26. A rotor 28 is arranged roughly centroid 30 in the tube pathway 22. A first lobe 32, a second lobe 34, and a third lobe 36 are arranged on the rotor 28.

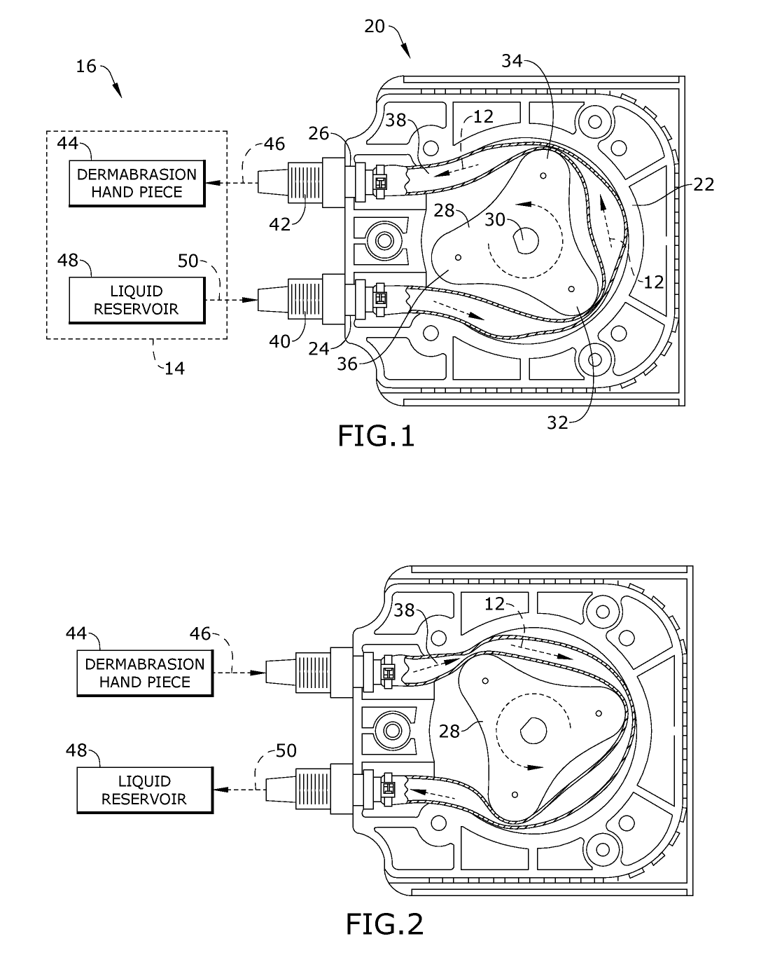

As shown in FIG. 1, the rotor 28 turning causes occlusion that traps the liquid 12 between the first lobe 32 and the second lobe 34 and moves the liquid 12 between the first opening 24 and the second opening 26. A tube 38 is joined to the first opening 24 with a first line connector 40. The tube 38 is further joined to the second opening 26 with a second line connector 42.

A hydro dermabrasion handpiece 44 is configured to administer the liquid 12 to a human. The hydro dermabrasion handpiece 44 is connected to the first line connector 40 with a first line 46. A liquid reservoir 48 is configured to store the liquid 12 for the hydro dermabrasion system. The liquid reservoir 48 is connected to the second line connector 42 with a second line 50.

A microcontroller is connected to the peristaltic pump 20 and is programmed with instructions. The microcontroller can engage the rotor 28 in a clockwise manner drawing liquid 12 from the liquid reservoir 48 and transferring the liquid 12 to the hydro dermabrasion handpiece 44.

The microcontroller can increase a speed of the rotor 28. This increases a volume of liquid 12 to the hydro dermabrasion handpiece 44. The microcontroller can decrease the speed of the rotor 28. This decreases the volume of liquid to the hydro dermabrasion handpiece 44.

The microcontroller can stop the rotor 28. This stops a volume of liquid 12 to the hydro dermabrasion handpiece 44. The microcontroller can engage the rotor 28 in a counterclockwise manner. This draws the liquid 12 from the first line 46 and transfers the liquid 12 to the liquid reservoir 48.

As used in this application, the term “a” or “an” means “at least one” or “one or more.”

As used in this application, the term “about” or “approximately” refers to a range of values within plus or minus 10% of the specified number.

As used in this application, the term “substantially” means that the actual value is within about 10% of the actual desired value, particularly within about 5% of the actual desired value and especially within about 1% of the actual desired value of any variable, element or limit set forth herein.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Any element in a claim that does not explicitly state “means for” performing a specified function, or “step for” performing a specified function, is not to be interpreted as a “means” or “step” clause as specified in 35 U.S.C. § 112, ¶6. In particular, any use of “step of” in the claims is not intended to invoke the provision of 35 U.S.C. § 112, ¶6.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A pumping system assembly, configured to adjust a flow rate of a liquid in a dermabrasion system;
a pump connected to the dermabrasion system, wherein the pump is configured to control to adjust the flow rate of the liquid in the dermabrasion system.

2. The pumping system assembly of claim 1, wherein a direction of fluid flow is controlled.

3. The pumping system assembly of claim 2, wherein the flow rate is deterministically controlled.

4. The pumping system assembly of claim 3, wherein a microcontroller controls the rate of the fluid flow.

5. The pumping system assembly of claim 1, wherein the flow rate is deterministically controlled.

6. The pumping system assembly of claim 5, wherein a microcontroller controls the flow rate.

7. The pumping system assembly of claim 1, wherein a microcontroller controls the flow rate.
